(19) United States
(12) Patent Application Publication
(10) Pub. No.: US 2008/0187563 A1
(43) Pub. Date: Aug. 7, 2008

(54) Oxygen delivery compositon

(76) Inventor: Ehud Levy, Suwanee, GA (US)

Correspondence Address: NEEDLE & ROSENBERG, P.C., SUITE 1000, 999 PEACHTREE STREET, ATLANTA, GA, 30309-3915, US

(21) Appl. No.: 11/982,809

(22) Filed: Nov. 5, 2007

Related U.S. Application Data
(60) Provisional application No. 60/857,752, filed on Nov. 7, 2006.
(60) Provisional application No. 60/967,528, filed on Sep. 5, 2007.

(51) Int. Cl. A61K 8/02; A61K 31/74; A61K 8/84; A61Q 17/04; A61Q 19/00

(52) U.S. Cl. 424/401; 424/78.02; 424/59; 424/78.03

(57) ABSTRACT

A composition is disclosed comprising a particle comprising the produce produced by the contacting of a silicate and a titanium containing compound, and at least one of a peroxide containing polymer, a cosmetically acceptable carrier, or a combination thereof. Also disclosed is a method for preparing a composition, comprising a) contacting an aluminum containing compound, an alkali metal silicate, and optionally a base to provide an aluminum silicate containing solution; b) contacting the solution prepared in a) with a titanium containing compound to provide a titanium aluminum silicate; c) exposing the solution produced in a) and/or the titanium aluminum silicate produced in b) to UV radiation; and then at least one of contacting at least a portion of the product of c) with at least one peroxide containing polymer, and/or contacting at least a portion of the product of c) with a cosmetically acceptable carrier.

### CROSS REFERENCE TO RELATED APPLICATIONS

This application claims priority to both U.S. Provisional Application Ser. No. 60/857,752, filed Nov. 7, 2006, and U.S. Provisional Application Ser. No. 60/967,528, filed Sep. 5, 2007, both of which are hereby incorporated by reference.

### FIELD OF THE INVENTION

This invention relates generally to an oxygen delivery composition, and specifically to a cosmetic composition that can deliver oxygen to a tissue of a subject.

### BACKGROUND OF THE INVENTION

Over the counter skin care and cosmetic compositions represent an extremely large segment of the consumer products industry. Moisturizer and other compositions intended to reduce wrinkles, soften, and smooth the skin surface are sold in large quantities to consumers who attempt to reduce damage and wrinkles resulting from aging and/or sun damage. While these products can provide moisturization, they are often without significant practical benefit or results with respect to reducing wrinkling or other effects of photodamage, because they do not address the intrinsic causes of wrinkling, such as, for example, weakening of collagen and elastin in the skin tissue, or the extrinsic causes of wrinkling, such as, for example, photodamage. Photodamage can be particularly severe in individuals taking certain medications, such as, for example, certain antiobiotics, or individuals who have undergone certain cosmetic procedures, such as, for example, microdermabrasion, laser resurfacing, or chemical peels, each of which has increased in popularity in recent years.

Botox injections can provide significant benefit in reducing wrinkling by causing small facial muscles to relax, but present an increased risk profile due to the need for repeated injections with muscle-paralyzing toxin. Hyperbaric oxygen chambers have also been used to attempt to reduce wrinkling, but are generally not practical for everyday use. Topical preparations containing vitamin A derivatives, such as tretinoin (e.g., RENOVA®, OrthoNeutrogena), can result in thinning of the skin and other undesirable effects. There remains a need in the art for a topically applied treatment that effectively reduces wrinkling and provides smoother skin, softer skin, or both.

There is a need to address the aforementioned problems and other shortcomings associated with traditional moisturizing and skin care products. These needs and other needs are satisfied by the cosmetic composition and treatment methods of the present invention.

### SUMMARY OF THE INVENTION

The present invention provides a cosmetic composition, and specifically a cosmetic composition that can deliver oxygen to a tissue of a subject.

In one aspect, the invention provides a composition comprising a particle comprising the product produced by the contacting of a silicate and a titanium containing compound, and at least one of a polymer, a cosmetically acceptable carrier, or a combination thereof.

In a second aspect, the invention provides a composition comprising a particle comprising the product produced by the contacting of a silicate and a titanium containing compound, and at least one of a peroxide containing polymer, a cosmetically acceptable carrier, or a combination thereof.

In a third aspect, the invention provides a composition comprising a cosmetically acceptable carrier and a peroxide containing polymer.

In a fourth aspect, the invention provides a method for preparing a composition, comprising a) contacting an aluminum containing compound, an alkali metal silicate, and optionally a base to provide an aluminum silicate containing solution; b) contacting the solution prepared in a) with a titanium containing compound to provide a titanium aluminum silicate; c) exposing the solution produced in a) and/or the titanium aluminum silicate produced in b) to UV radiation; and then d) at least one of contacting at least a portion of the product of c) with at least one peroxide containing polymer, and/or contacting at least a portion of the product of c) with a cosmetically acceptable carrier.

In a fifth aspect, the invention provides a method for enhancing the appearance of skin in a subject, comprising contacting a tissue of the subject in need of such enhancement with a sufficient quantity of any of the compositions described above.

In a sixth aspect, the invention provides a kit comprising a particle comprising the product produced by the contacting of a silicate and a titanium containing compound, a peroxide containing polymer, and a cosmetically acceptable carrier.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

The accompanying drawings, which are incorporated in and constitute a part of this specification, illustrate several aspects of the invention and together with the description, serve to explain the principles of the invention.

### BRIEF DESCRIPTION OF THE DRAWINGS

FIGS. 1A and 1B are photographs of a subject both before and after repeated applications of the inventive composition, respectively, illustrating a reduction in the appearance of wrinkles in the subject, in accordance with various aspects of the present invention.

FIG. 2 is a graph of senescence associated beta galactosidase activity for the following samples of BJ human dermal fibroblast cells upon repeated exposure to UVB radiation: an untreated control sample (CTL) not exposed to UVB radiation, an untreated sample exposed to UV-B radiation (UVB), samples treated with 10 μg/ml, 100 μg/ml, and 200 μg/ml of the inventive composition, an internal control, and a Trolox vitamin-E analogue internal control. The mean proportion and standard deviation (denoted by error bars) of positive cells are represented for each sample.

FIG. 3 is a graph of intracellular reactive oxygen species production for the following samples of BJ human dermal fibroblast cells upon repeated exposure to UVB radiation: an untreated control sample (CTL) not exposed to UVB radiation, an untreated sample exposed to UV-B radiation (UVB), samples treated with 10 μg/ml, 100 μg/ml, and 200 μg/ml of the inventive composition, an internal control, and a Trolox vitamin-E analogue internal control.

### DETAILED DESCRIPTION OF THE INVENTION

The present invention may be understood more readily by reference to the following detailed description of preferred aspects of the invention and the Examples included therein and to the Figures and their previous and following description. It is also to be understood that the terminology used herein is for the purpose of describing particular aspects only and is not intended to be limiting.

It must be noted that, as used in the specification and the appended claims, the singular forms “a,” “an” and “the” include plural referents unless the context clearly dictates otherwise. Thus, for example, reference to “an aromatic compound” includes mixtures of aromatic compounds.

Often, ranges are expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

In this specification and in the claims that follow, reference will be made to a number of terms that shall be defined to have the following meanings:

A weight percent of a component, unless specifically stated to the contrary, is based on the total weight of the formulation or composition in which the component is included.

“Optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyl where there is substitution.

As briefly introduced above, the present invention provides a cosmetic composition and a method of treating a tissue of a subject. In one aspect, the present invention provides a composition comprising a cosmetically acceptable carrier and a polymer. In other aspect, the present invention provides a composition comprising a cosmetically acceptable carrier and a peroxide containing polymer. In yet other aspects described in detail below, the inventive composition comprises a peroxide complexed hydroxyl titanium aluminum silicate particle comprising a silicate and a titanium compound that have been contacted, and a peroxide containing polymer.

In one aspect, the invention provides a cosmetic composition that can be applied to, for example, the skin of a subject and that can provide one or more cosmetic benefits thereto, such as, for example, increasing the amount of oxygen supplied to the skin, improved appearance, decreased and/or preventing wrinkling, or a combination thereof.

### Particles

The particles of the present invention can be particles made from a silicate and titanium compound. Typically, they are suitable for use in delivering oxygen to a tissue of a subject.

In one aspect, the particles of the invention can be produced by a method, such as, for example, that described in U.S. Pat. No. 5,612,522 and/or U.S. Pat. No. 7,288,498, the entire contents of which are hereby incorporated by this reference. In such an aspect, an aluminosilicate gel is formed from a slurried aluminum hydrate and an alkali metal silicate in the presence of a base, such as, for example, an aqueous sodium hydroxide in the presence of ultraviolet (UV) radiation having a wavelength of, for example, from about 2000 Å to about 3,900 Å. In another aspect, particles are prepared by first contacting alumina with an aqueous sodium hydroxide solution. The resulting mixture can optionally be heated until the alumina is substantially dissolved.

A separate aqueous solution of sodium silicate (e.g., PQ Water Glass, available from PQ Corporation, Valley Forge, Pa., USA) and a source of titanium, such as titanium dioxide, can be prepared and combined with the first solution or gel of alumina. The resulting slurry or gel can optionally be mixed to ensure that the components are substantially or thoroughly mixed.

The resulting slurry or gel can then be exposed to UV radiation. Exposure of the resulting slurry or gel to UV radiation can, in various aspects, result in an increase in the hardness of particles that can be disposed within the slurry or gel. In one aspect, the increase in hardness is substantial and is detectable by visual inspection and/or touch. The particular wavelength and/or intensity of UV radiation to which the resulting slurry or gel is exposed is not important. The resulting slurry or gel can optionally be spread and/or dispersed in a manner that all or substantially all of the slurry or gel is exposed to the UV radiation. If spread and/or dispersed, the particular manner in which the slurry or gel is spread and/or dispersed can vary, depending upon, for example, the intensity of the UV radiation. Exposure of the slurry or gel to UV radiation can occur simultaneous to or subsequent to the addition of the titanium containing component, such as, for example, titanium dioxide.

The UV radiation can be any suitable UV radiation and can be derived from or emitted by any suitable source. In one aspect, the source of UV radiation is natural sunlight. In another aspect, the source of UV radiation is a commercially available germicidal UV lamp. In other aspects, the source of UV radiation can be any radiation source capable of providing UV radiation, such as for example, fluorescent, infrared, and/or incandescent radiation sources.

The temperature and time of UV exposure can also vary, depending upon, for example, the thickness of the slurry or gel and/or the wavelength and intensity of the UV radiation. In various aspects, the time of exposure to UV radiation can be from about 5 to about 14 days, for example, about 1, 2, 3, 4, 5, 7, 9, 12, or 14 days. In other aspects, the exposure to UV radiation can be less than 5 or greater than 14 days. In a specific aspect, the slurry or gel is spread to a thickness of about 2 inches and then exposed to UV radiation from a germicidal lamp for a period of about 3 days. In another aspect, the exposure to UV radiation is performed at a temperature of from about 75° F. to about 90° F., preferably about 90° F.

The titanium containing compound useful in the invention can be a titanium dioxide, titanium hydroxide, or other titanium containing compound. In one aspect, the titanium containing compound is a titanium dioxide, such as, for example, a fumed or plasma generated titanium dioxide. In a specific aspect, the titanium containing compound is a fumed titanium dioxide. In another specific aspect, the titanium containing compound is a plasma generated titanium dioxide. The particle size of the titanium containing compound can vary. In one aspect the particle size of the titanium containing compound can be from about 25 nm to about 200 nm, such as, for example, about 25, 50, 75, 100, 125, 150, 175, or 200 nm. It should be understood that size of particles, such as titanium containing compounds, are usually distributional, and thus, the mean particle size and distribution of a given sample of particles can vary. The present invention is not intended to be limited to a particular particle size and/or particle size distribution. In a specific aspect, the mean particle size of a titanium containing compound is about 50 nm. In another specific aspect, the mean particle size of a titanium containing compound is about 125 nm.

The silicate useful in the invention can be any suitable silicate for forming the inventive composition, such as, for example, an aluminosilicate, a sodium silicate, a transition metal silicate, or a combination thereof.

The amount of the titanium compound contained within the particle composition can vary and can, in various aspects, generally be maintained at a level less than or equal to about 10 wt. %, such as for example, from about 0.1 wt. % to about 10 wt. %, or about 10, 9, 8, 7, 6, 5, 4, 3, 2, 1, or 0.1 wt. %; more desirably at a level less than or equal to about 8 wt. %, such as, for example, from about 0.1 wt. % to about 8 wt. %, or about 8, 7, 6, 5, 4, 3, 2, 1, or 0.1 wt. %; or even more desirably at a level less than or equal to about 4 wt. %, such as for example, from 1 wt. % to 4 wt. %, or about 4, 3, 2, or 1 wt. %. In various specific aspects, the amount of titanium compound within the particle composition is about 2 wt. %, about 3 wt. %, or about 4 wt. %.

The resulting particulate material can be described as a hydroxyl titanium aluminum silicate. The specific composition of the resulting material can vary due to factors, such as, for example, the bulk density of one or more components. In various aspects, the resulting composition can contain from about 0.1 wt. % to about 10 wt. % by weight of the titanium compound, such as titanium dioxide and/or titanium hydroxide, and from about 90% to about 98% aluminosilicate, can then optionally be washed, dried, and/or screened to a desired particle size distribution. Drying, if performed, can be performed in a static or flowing air atmosphere or under any other suitable atmosphere, such as for example, an air atmosphere comprising from about 5 to about 10 vol. % nitrogen or a mixture of ammonia and air. The time and temperature of an optional drying step can also vary. In one aspect, the optional drying step, if performed, can be at a temperature of from about 200° C. to about 1,200° C. In one specific aspect, such an optional drying step can be performed at about 500° C. for a period of time from about 2 to about 24 hours.

The mean particle size can vary from about 0.1 μm to about 5 mm, but in various aspects, the mean particle size can be less than 0.1 μm or larger than 5 mm, and as such, the present invention is not intended to be limited to a particular particle size. In a specific aspect, the mean particle size is about 20 μm. It should be appreciated that the size of any individual or group of particles can vary and can change over time, depending upon, for example, environmental factors. For example, if particles are mixed in a formulation, such as a cream or serum, the mean particle size can decrease with continued contact with the formulation. The desired particle size distribution, if one exists, can vary depending upon the intended application of the composition. In one aspect, the particle size distribution has an average granular particle size ranging from about 1 mm to about 5 mm. In another aspect, the particle size distribution has an average powder particle size distribution of from about 1 μm to about 500 μm.

The particles can comprise a single composition and/or particle size distribution or can comprise one or more composition and/or particle size distribution. It is not necessary that all particles comprise the same composition. In one aspect, all or substantially all particles comprise the same or similar composition. In another aspect, the particles comprise at least two or more varying compositions.

Alumina, aluminosilicates, titanium dioxide, and the other materials described herein for the production of titanium silicate particles are commercially available and one of skill in the art could readily select appropriate starting materials for use in the production of titanium silicate particles.

### Polymer

The polymer of the present invention can comprise any suitable polymer that is capable of containing or closely associating with a peroxide material. The polymer can comprise a single polymer, a mixture of polymers, and/or a copolymer. It is not necessary that a polymer be a peroxide-containing polymer and the present invention is limited to aspects in which the polymer is a peroxide-containing polymer. In one aspect, the polymer does not contain and is not closely associated with a peroxide material. In another aspect, the polymer is a peroxide-containing polymer. In any of the aspects described herein where a peroxide containing polymer is recited, aspects comprising a polymer not including a peroxide material are also envisioned and considered to be a part of the invention. Similarly, in aspects where a polymer is recited, aspects comprising a peroxide-containing polymer are also envisioned and considered to be a part of the invention.

In one aspect, the polymer is a peroxide containing polymer, such as, for example, a polyvinylpyrrolidone (“PVP”) that has been treated with hydrogen peroxide. In another aspect, the polymer is a copolymer of a polyvinylpyrrolidone and a comonomer, such as a vinyl acetate that has been treated with hydrogen peroxide. In an exemplary aspect, the polymer is a copolymer, such as PLASDONE® S-630 treated with about 30 wt. % hydrogen peroxide, available from International Specialty Products. In another aspect, the polymer comprises at least one hydrogen bonded complex of a linear, vinyl pyrrolidone homopolymer and hydrogen peroxide, such as, for example, PEROXYDONE® K30, K90, or a hydrogen bonded complex of a crosslinked polyvinylpyrrolidone and hydrogen peroxide, such as, for example, XL-10, available from International Specialty Products.

The peroxide material of the present invention can comprise a hydrogen peroxide, an organic peroxide, or a reaction product and/or residue of a hydrogen peroxide, an organic peroxide, and/or a polymer. The amount of peroxide can vary depending on the specific materials used and the intended application. In various aspects, the polymer component can comprise from about 5 wt. % to about 60 wt. %, for example, about 5, 7, 9, 14, 18, 20, 24, 28, 30, 34, 38, 42, 44, 48, 50, 53, 57, or 60 wt. % of the peroxide component or material; preferably from about 10 wt. % to about 30 wt. % of the peroxide component, such as, for example, about 10, 12, 14, 16, 18, 20, 22, 24, 26, 28, or 30 wt. % peroxide component. In other aspects, the amount of the peroxide component can be less than 10 wt. % or greater than 30 wt. % of the polymer component. Polymers, peroxide materials, and peroxide containing polymers are commercially available and one of skill in the art could readily select an appropriate polymer, peroxide material, and/or peroxide containing polymer for use in the various aspects of the present invention. The peroxide containing polymer should be compatible with the particle of the invention.

Application of Polymer and/or Cosmetically Acceptable Carrier

The polymer, such as, for example, a peroxide containing polymer, can be contacted with and/or mixed with the titanium silicate particles. The polymer can be in contact with some or all of the particles and it is not necessary that the polymer be in contact with all of the particles.

The amount of peroxide containing polymer can vary depending on factors, such as the specific composition, bulk density of particulate materials, and the intended application. In various aspects, the amount of peroxide containing polymer can be from about 0.1 to about 40 wt. %, for example, about 0.1, 0.5, 1, 2, 3, 5, 7, 10, 12, 14, 18, 20, 22, 25, 28, 30, 35, or 40 wt. %; preferably from about 0.1 wt. % to about 10 wt. %, for example, about 0.1, 0.5, 1, 2, 3, 5, 7, or 10 wt. % of the total composition. In one aspect, the peroxide containing polymer is about 6 wt. % of the total composition. In another aspect, the peroxide containing polymer is about 10 wt. % of the total composition. The specific amount of peroxide containing polymer can vary depending on the intended application and can, in various aspects, be less than 0.1 or greater than 25 wt. %.

The peroxide containing polymer can be contacted with the particles in any suitable manner, such as, for example, mixing, coating, or spraying. The conditions, such as, for example, time and temperature, of contacting can also vary depending on the specific technique and intended composition and application. In one specific aspect, the peroxide containing polymer is blended with the particles for about 10 minutes to produce a composition comprising about 18 wt. % of the peroxide containing polymer. In another specific aspect, the peroxide containing polymer is applied as a micro-coating on the surface of the particles through the use of an air jet mixing technique. In a preferred aspect, the peroxide containing polymer is coated on at least a portion of the particles.

The resulting composition of the particles and peroxide containing polymer can be used or incorporated as produced, or can be subsequently processed to a desired particle size for incorporation into, for example, a cream, spray, gel, serum, paste, or other suitable cosmetic and/or personal care product.

The peroxide containing polymer can be mixed with the particles and/or a cosmetically acceptable carrier, such as, for example, a serum, in any order. The peroxide containing polymer can be mixed with the particles at the time of manufacture and/or at any time up to the intended use of application. In one aspect, the peroxide containing polymer is contacted with and/or mixed with the particles at manufacture and the resulting peroxide/particle mixture can be subsequently mixed a cosmetically acceptable carrier, such as, for example, a serum. In another aspect, the particles can be mixed with a cosmetically acceptable carrier, such as, for example, a serum, and the resulting product then mixed with the peroxide containing polymer. In yet another aspect, each of the particles, peroxide containing polymer, and cosmetically acceptable carrier can be maintained separately and mixed at or just prior to the intended use or application. If the peroxide containing polymer is mixed with either the particles, the cosmetically acceptable carrier, or a combination thereof, it is preferred that the moisture content of the particles and/or the cosmetically acceptable carrier be minimal so as to prevent premature degradation of the peroxide. If the peroxide containing polymer is mixed with a moisture containing component at a time just prior to, for example, about 0.1, 0.5, 1, 2, 4, 8, 12, or 24 hours, the intended use or application, any degradation of the peroxide containing component that occurs, if any, should be negligible and would not adversely affect the performance of the composition. In various aspects, the particles, peroxide containing polymer, and cosmetically acceptable carrier can be provided in a kit. Such a kit can comprise any combination of the components described herein. In one specific aspect, a kit comprises a first material comprising the particles and a cosmetically acceptable carrier, and a second material comprising a peroxide containing polymer. In a second specific aspect, a kit comprises a first material comprising the particles and a peroxide containing polymer, and a second material comprising a cosmetically acceptable carrier. In a third specific aspect, a kit comprises a first material comprising the particles, a second material comprising a cosmetically acceptable carrier, and a third material comprising a peroxide containing polymer. While not wishing to be bound by theory, the presence of moisture either in or on the surface of the tissue to which the composition is applied, or in any of the components with which the peroxide containing polymer is mixed can be beneficial in promoting the release of decomposition of the peroxide component and the delivery of oxygen to the tissue, provided that such contact with moisture occurs just prior to the intended use and/or application of the composition.

In other aspects, a peroxide containing polymer, such as, for example, a polyvinylpyrrolidone and/or a vinylpyrrolidone complexed with a hydrogen peroxide, can be contacted with a cosmetically acceptable carrier in the absence of a particle. Such a composition can be prepared and optionally applied, as described herein, to treat a tissue of a subject.

The composition can also be encapsulated for delayed release using techniques well known in the cosmetic care and/or pharmaceutical arts. In various aspects, the particles can be processed, such as grinding, prior to, during, or subsequent to contacting with a peroxide containing polymer.

### Other Components

The composition of the present invention can also comprise other components, provided that such optional components do not adversely react with or affect the ability of the composition to deliver oxygen to a tissue of a subject. Such optional components can comprise such materials typically found in cosmetic and/or personal care products such as sunscreens, make-up, moisturizing creams, and/or bath soaks. An optional component can, in various aspects, be a cosmetically acceptable carrier that can provide a formulation such as a paste, cream, lotion, or bath soak. The composition of the present invention and any optional components can be present in any suitable form, such as for example, an emulsion, a suspension, or an encapsulated form. Optional components are known and one of skill in the art could readily select any desired optional components for a particular formulation or application.

### Method of Treating

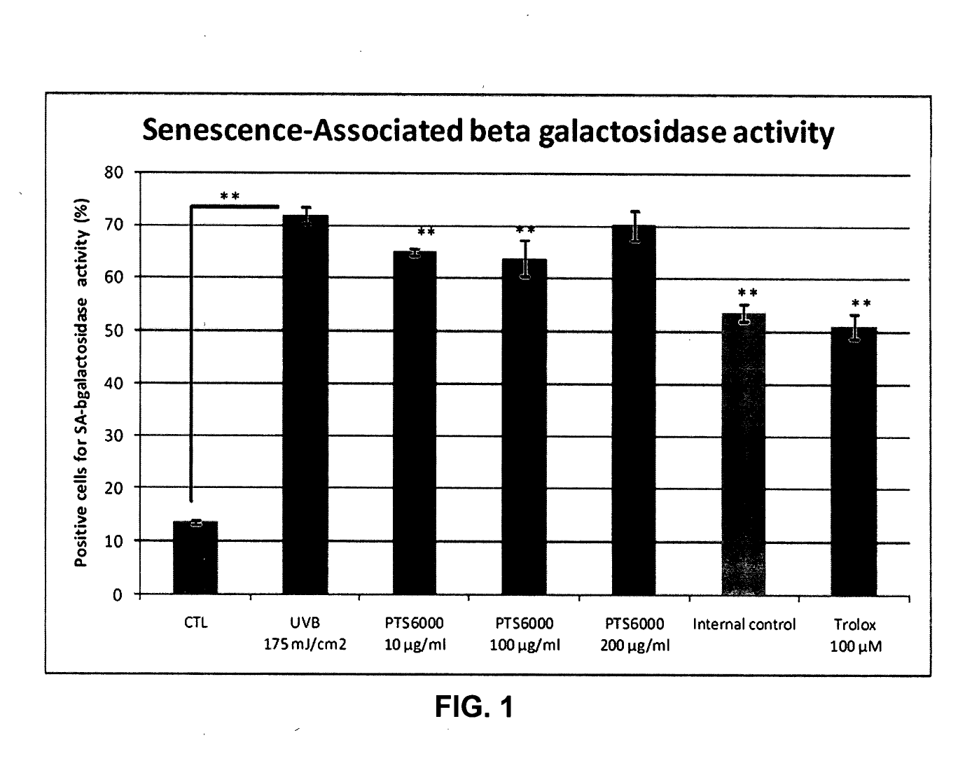

When applied to a tissue of a subject, the composition of the present invention can improve the appearance of the tissue by, for example, reducing the amount of (e.g., size, number, and/or depth) or eliminating visible wrinkles in the tissue. FIGS. 1A and 1B illustrate photographs of a subject both before and after repeated applications of the inventive composition. The appearance of wrinkles on the subject's face is visibly reduced after such repeated applications of the inventive composition. Such wrinkles can result from aging, photodamage, and/or other physical or medical conditions. The tissue can be any suitable tissue of the subject, such as the face, cheek, nose, ear, arm, leg, body, scalp, or any other skin or tissue of the subject. The subject can be any subject having a skin that wrinkles, such as a mammal, such as a human. The composition of the present invention can also be applied to the tissue of a subject to prevent wrinkles and/or reduce the intensity of wrinkles not yet formed.

The composition can be applied to a tissue in any suitable amount to achieve a desired result. Typically, the composition is applied directly to the external tissue surface. In various aspects, the composition can be applied in an amount ranging from about 0.01 g to about 5 g of the composition per application. If the composition is mixed in a formulation of, for example, a personal care product, the dosage can vary depending on, inter alia, the concentration of the composition in the personal care product. In various aspects, a quantity of from about 0.01 g to about 5 g, for example, about 0.01, 0.02, 0.05, 0.1, 0.2, 0.3, 0.5, 0.7, 0.9, 1, 1.2, 1.4, 1.6, 1.8, 2, 2.5, 3, 3.5, 4, 4.5, 4.9, or 5 g of the composition can be mixed with a quantity of from about 0.5 ml to about 10 ml, for example, about 0.5, 0.8, 1, 1.5, 2, 3, 4, 5, 6, 7, 8, 9, or 10 ml of a formulation, such as, for example, a serum. In various aspects, a quantity of less than 0.01 g or greater than 5 g of the composition can be mixed with a quantity of less than 0.5 ml or greater than 10 ml of a formulation and the present invention is not intended to be limited to a particular concentration range. In another aspect, the concentration of the composition in a formulation is any concentration sufficient to provide the desired result, such as, for example, wrinkle reduction. In one specific aspect, a quantity of about 0.01 g of the composition is mixed with about 10 ml of a serum. In another specific aspect, about 2 g of the composition is mixed with about 5 ml of a serum. In yet another specific aspect, about 5 g of the composition is mixed with about 0.5 ml of a serum.

While not wishing to be bound by theory, it is believed that the titanium and silicate component of the composition promotes or enhances the oxidative effect of the peroxide delivered by the peroxide containing polymer component, thus increasing the release and/or delivery of oxygen over that of the polymer and/or peroxide alone. When used in any of the various aspects described herein, the inventive composition can deliver oxygen and/or an oxygen containing compound to at least a portion of the tissue of a subject at a depth of up to about 0.8 mm or more from the tissue surface. The time period over which oxygen and/or an oxygen containing species is delivered to a tissue or a portion thereof can vary depending on factors such as the concentration of the peroxide containing polymer, particle size, concentration of the composition in a particular formulation, and amount and rate of application of a formulation. The delivery of such species is typically slow, occurring over a period of hours.

When used in, for example, a cosmetic formulation ranging from about 0.1 wt. % of the cosmetic composition formulation to about 100 wt. % of the formulation, the compositions of the invention can reduce observable wrinkles in some areas of the body by as much as 70%. Again, not wishing to be bound by theory, it is believed that the combination of a titanium and silicate particle and peroxide-containing polymer results in delivery of oxygen to the capillaries and other blood vessels supplying the skin and adjacent tissues, and that this has a positive effect on wrinkle reduction, in similar manner to that of a hyperbaric oxygen chamber treatment. Application of the inventive composition to the tissue of a subject can also result in other benefits, such as, for example, the closing and/or reduction in size of pores and the reduction and/or elimination of infections on or in the tissue.

### Performance

The oxygen delivery activity of a composition prepared in accordance with various aspects of the present invention can be measured in any suitable manner, such as for example, oxidation of an organic indicator.

In one aspect, the oxygen delivery activity of a composition is measured by mixing a quantity of the composition with an amount of an organic indicator. In one aspect, the organic indicator is a dye that can be oxidized and that will lose at least a portion of its optical absorbance upon oxidation. In a specific aspect, the organic indicator is a crystal violet solution. The composition and organic indicator can be prepared and/or mixed in solution, such as, for example, in aqueous solutions. After mixture, the time required for all or a pre-determined portion of the color (e.g., optical absorbance) of the organic indicator to diminish or disappear can be recorded. In one aspect, all or substantially all of the optical absorbance of the organic indicator should be eliminated within about 24 hours after contact with the composition.

In an exemplary aspect, a composition of the invention comprising about 2 wt. % peroxide was able to quickly oxidize the organic indicator. In application to a tissue of a human subject, such a concentration of peroxide in the inventive composition did not result in burning of the subject's skin. In comparative tests, samples of titanium dioxide, titanium dioxide photocytilst, zeolite, titanium silicate (ENGELHARD), polyvinylpyrrolidone, and polyvinylprrolidone peroxide all failed to show a reduction in the intensity of color (optical absorbance) of the organic indicator.

Although several aspects of the present invention have been illustrated and described in the detailed description, it should be understood that the invention is not limited to the aspects disclosed, but is capable of numerous rearrangements, modifications, and substitutions without departing from the spirit of the invention as set forth and defined by the following claims.

### EXPERIMENTAL

The following examples are put forth so as to provide those of ordinary skill in the art with a complete disclosure and description of how the cosmetic composition and associated processes and methods are constructed, used, and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. (Celsius) or is at ambient temperature, and pressure is at or near atmospheric.

### Example 1

### Preparation of Titanium Silicate

In a first example, a titanium silicate material was prepared by contacting approximately 500 g of alumina (710, available from Selecto) with approximately 480 ml of 50% aqueous sodium hydroxide. The alumina and sodium hydroxide were mixed and heated until the alumina substantially dissolved, at a temperature of approximately 160° F. A separate solution was prepared by thoroughly mixing 9 L of deionized water, and 3 L of sodium silicate (PQ Water Glass, PQ Corporation, Valley Forge, Pa., USA). 40 g of approximately 40 nm titanium dioxide (P25) was then added to the separate solution with additional thorough mixing at high speed (approximately 3,900 rpm). The two solutions were then combined and mixed thoroughly. The resulting gel/slurry was spread out such that it was less than about 2 inches thick, and subjected to UV curing under conventional germicidal UV lamps at temperatures of between 75° F. and 110° F. for about 3 days.

### Example 2

### Preparation/Treatment Titanium Silicate

In a second example, the material prepared in Example 1 was treated by drying in an oven under an air atmosphere comprising about 5-10 vol. % nitrogen at a temperature of approximately 500° C. and a gas flow rate of approximately 300 cc per minute for about 24 hours.

### Example 3

### Preparation/Treatment Titanium Silicate

In a third example, a titanium dioxide (P25) powder is treated. Titanium dioxide powder having an average particle size of about 40 nm was heated under atmospheric conditions in the presence of nitrogen at a temperature of about 700° C. for 24 hours, and subsequently cooled.

### Example 4

### Application of Peroxide Containing Polymer

In a fourth example, the titanium silicate material (˜72 wt. %) prepared in Example 2 was combined with the titanium powder (˜10 wt. %) produced in Example 3 in a ribbon mixer, slowly blended to prevent heat generation. The resulting mixture was then coated with a complex of hydrogen peroxide and PVP (PEROXYDONE® K30, ISP) in an amount of 18 wt. % (all percentages based on the weight of the final composition) by blending for about 10 minutes.

### Example 5

### Analysis of Oxygen Delivery Activity

In a fifth example, the oxygen generating activity of a composition was determined using a Crystal Violet Blue indicator. Approximately 1.0 g of powder (comprising about 65 wt. % titanium silicate material and about 35 wt. % hydrogen peroxide complex of polyvinylpyrrolidone equivalent to about 2 wt. % peroxide) was mixed with approximately 200 ml of deionized water. The mixture was stirred and then 1.0 ml of an aqueous 2 wt. % solution of Crystal Violet (hexamethyl violet) indicator. After addition, the time required for the blue color of the Crystal Violet solution to disappear was recorded. The time required for the color of the organic indicator to disappear when combined with the inventive composition was 5 min. In comparison, similar tests conducted on titanium dioxide, titanium dioxide photocytilst, zeolite, titanium silicate (ENGELHARD), polyvinylpyrrolidone peroxide, and polyvinylpyrrolidone alone all failed to show a reduction in the color of the organic indicator.

### Example 6

### Determination of Senescence Associated Beta Galactosidase Activity

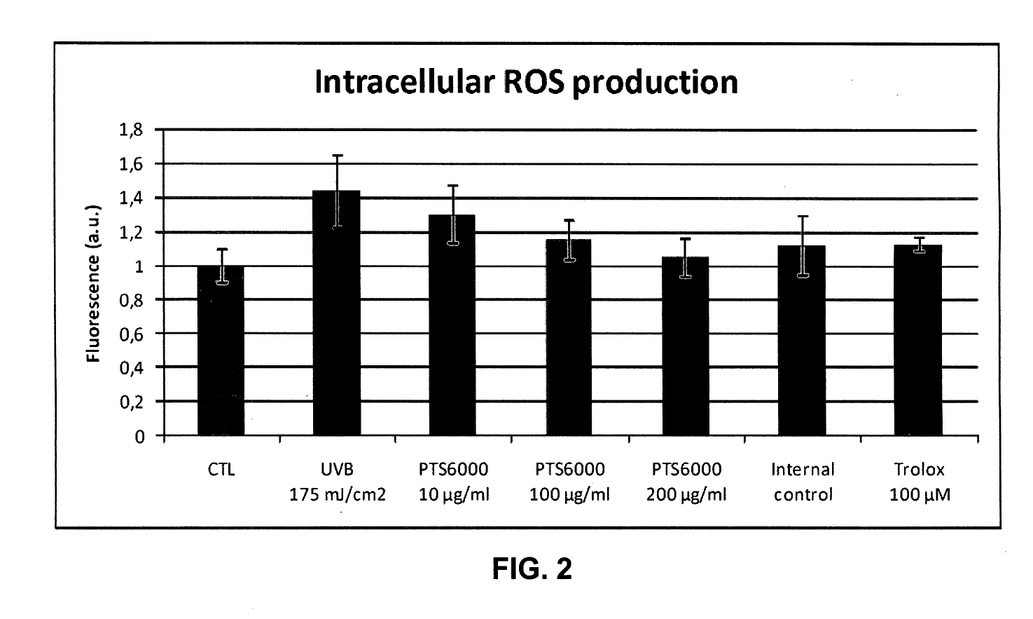

In a sixth example, senescence associated beta galactosidase (SA β-gal) activity was determined for a variety of BJ human dermal fibroblast samples on exposure to UV radiation. SA β-gal activity can be a reliable marker of fibroblast senescence. As such, analysis of SA β-gal activity upon exposure to UV radiation can provide information related to a fibroblast's ability to withstand UV radiation without premature ageing and/or senescence. The BJ dermal fibroblasts were classically grown in DMEM/M199 (Dulbecco's Modified Eagle's Medium/medium 199 hank's HEPES, Invitrogen, UK), 10% FCS (fetal calf serum, Invitrogen, UK), and 100 μg/ml penicillin/streptomycin. The fibroblast samples included: an untreated control sample (CTL) not exposed to UVB radiation; an untreated control sample (UVB) exposed to UVB radiation; three samples treated with the inventive composition (PTS6000) at 10 μg/ml, 100 μg/ml, and 200 μg/ml, respectively; an internal control; and a Trolox (vitamin E analogue) control. Each sample was stressed by five exposures to UVB radiation at 175 mJ/cm² per exposure. The effect of UVB stress induced premature senescence on each cell sample was determined 72 hours after the last stress. Cells were trypsinised and seeded at a density of 10,000 cells per well in 6-well plates containing 2 ml of DMEM/M199 and 10% FCS and senescence activity was tested 24 hours later. The population of SA β-gal positive cells was determined by counting 400 cells per dish using a double blind procedure. Triplicate analyses were performed for each sample. UVB stressed cells were characterized by a major proportion of enlarged cells typical of senescent cultures. The proportions of cells positive for SA β-gal activity, expressed as a percentage of the total number of cells counted in each dish, are illustrated in FIG. 2. For cells exposed to UVB radiation and treated with the small doses of the inventive composition in the culture medium, a reduction in the appearance of SA β-gal positive cells was observed as compared to untreated control samples. This reduction in the appearance of SA β-gal positive cells suggests that application of the inventive composition can prevent and/or reduce cell senescence from UV radiation.

### Example 7

### Determination of Intracellular Reactive Oxygen Species

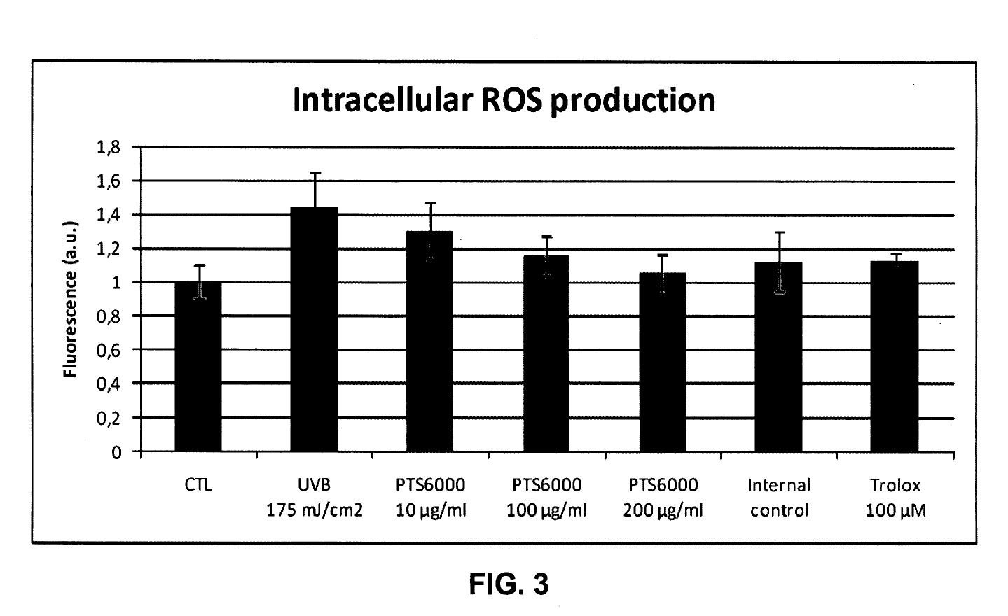

In a seventh example, the effect of application of the inventive composition to a series of BJ human dermal fibroblast samples (as described in Example 6) on intracellular levels of reactive oxygen species was determined. Free radicals and reactive oxygen species (ROS) caused by oxidative stress, exposure to UVB radiation, or other environmental factors can cause cellular damage and aging. An oxidant sensitive probe, H₂DCFDA (2′,7′-dichlorodihydrofluorescein acetate) was used to determine the intracellular levels of ROS in the cell sample described above. H₂DCFDA is a fluorogenic probe for ROS that is nonfluorescent until the acetate groups of the probe are removed by intracellular esterases and oxidation occurs within the cell. Each of the cell samples were grown and prepared as described in Example 6, including exposure to UVB radiation and treatment with the inventive composition. The results of these analyses are illustrated in FIG. 3. Exposure to UVB radiation produced a sustained level of ROS for a significant period of time after the last irradiation in untreated samples. In contrast, samples treated with the inventive composition exhibited significantly lower levels of ROS as compared to untreated samples. These results suggest that the application and/or presence of the inventive composition can prevent and/or reduce the generation of ROS on exposure to UV radiation.

Throughout this application, various publications are referenced. The disclosures of these publications in their entireties are hereby incorporated by reference into this application.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising:
a) a particle comprising the product produced by the contacting of a silicate and a titanium containing compound, and
b) at least one of a polymer, a cosmetically acceptable carrier, or a combination thereof.

2. The composition of claim 1, wherein at least one of a polymer is present, and wherein the at least one polymer comprises a peroxide-containing polymer.

3. The composition of claim 2, wherein the peroxide-containing polymer comprises hydrogen peroxide and at least one of polyvinylpyrrolidone, vinyl pyrrolidone, or a combination thereof

4. The composition of claim 2, wherein the peroxide-containing polymer comprises a mixture of polyvinylpyrrolidone (PVP) and hydrogen peroxide.

5. The composition of claim 2, wherein the peroxide-containing polymer comprises from about 10 wt. % to about 30 wt. % of a peroxide component.

6. The composition of claim 2, wherein the peroxide-containing polymer comprises from about 0.1 wt. % to about 40 wt. % of the composition.

7. The composition of claim 1, wherein the silicate comprises an aluminum silicate.

8. The composition of claim 1, wherein the titanium containing compound comprises a titanium dioxide.

9. The composition of claim 1, wherein the particle comprises a hydroxyl titanium silicate particle.

10. The composition of claim 1, wherein a polymer is present, and wherein the polymer at least partially coats at least a portion of the particles.

11. The composition of claim 1, comprising both a polymer and a cosmetically acceptable carrier.

12. The composition of claim 1, wherein the titanium containing compound is present in an amount ranging from about 1 wt. % to about 4 wt. % of the weight of the particles.

13. The composition of claim 1, wherein the composition is in the form of a paste, serum, cream, lotion, bath soak, or a combination thereof.

14. The composition of claim 1, wherein the composition is in the form of a sunscreen.

15. The composition of claim 1, wherein the composition is in the form of a cosmetic make-up.

16. A kit comprising:
a) a particle comprising the product produced by the contacting of a silicate and a titanium containing compounds,
b) a polymer, and
c) a cosmetically acceptable carrier.

17. The kit of claim 19, wherein the polymer comprises a peroxide containing polymer.

18. The kit of claim 19, wherein the particle comprises a hydroxyl titanium silicate particle.

19. A composition comprising:
a) a cosmetically acceptable carrier, and
b) a peroxide containing polymer.

20. A method for preparing a composition, comprising:
a) contacting an aluminum containing compound, an alkali metal silicate, and optionally a base to provide an aluminum silicate containing solution;
b) contacting the solution prepared in a) with a titanium containing compound to provide a titanium aluminum silicate;
c) exposing the solution produced in a) and/or the titanium aluminum silicate produced in b) to UV radiation; and then
d) at least one of:
i) contacting at least a portion of the product of c) with at least one peroxide containing polymer; and/or
ii) contacting at least a portion of the product of c) with a cosmetically acceptable carrier.

21. The method of claim 20, wherein step d) comprises: (i) contacting at least a portion of the product of c) with a cosmetically acceptable carrier, and then (ii) contacting at least a portion of the product of (i) with at least one peroxide containing polymer.

22. The method of claim 20, wherein the peroxide containing polymer is present and comprises hydrogen peroxide and at least one of polyvinylpyrrolidone, vinyl pyrrolidone, or a combination thereof, and wherein the titanium containing compound comprises a titanium dioxide.

23. A method for enhancing the appearance of skin in a subject, comprising contacting a tissue of the subject in need of such enhancement with a sufficient quantity of the composition of claims 1.

24. The method of claim 23, wherein the enhancing comprises reducing the appearance of wrinkles, photodamage, or a combination thereof.

25. The method of claim 23, wherein the tissue comprises at least one of face, cheek, nose, ear, arm, leg, body, scalp, or a combination thereof of a human subject.
